(19) United States
(12) Patent Application Publication
(10) Pub. No.: US 2021/0346119 A1
(43) Pub. Date: Nov. 11, 2021

(54) AEROSOL EVACUATION SYSTEM

(71) Applicants: Edward E. WHITE, Elizabethtown, PA (US); James D. REIDER, Elizabethtown, PA (US); John Trevor REIDER, Elizabethtown, PA (US)

(72) Inventors: Edward E. WHITE, Elizabethtown, PA (US); James D. REIDER, Elizabethtown, PA (US); John Trevor REIDER, Elizabethtown, PA (US)

(21) Appl. No.: 17/230,510

(22) Filed: Apr. 14, 2021

Related U.S. Application Data
(60) Provisional application No. 63/020,354, filed on May 5, 2020.

(51) Int. Cl. A61B 90/40; A61L 9/20; A61L 9/12

(52) U.S. Cl. CPC A61B 90/40; A61L 9/20; A61B 2090/401; A61L 2209/14; A61L 2209/15; A61L 9/122

(57) ABSTRACT

A system for removing potentially harmful aerosols for protecting medical or dental practitioners and patients from contaminants including a treatment space and a conduit extending from the treatment space to a plenum space. The conduit includes a suspended collection arm extending into the treatment space. The collection arm is movable to a position adjacent a source of aerosols. The system further includes an exhaust fan assembly arranged and disposed along the conduit to provide movement of air therethrough. An air treatment unit is arranged and disposed along the conduit to draw air through the conduit, filter the air and treat the air prior to discharging the air into the plenum space

### FIELD OF THE INVENTION

The present invention is directed to a system and method for removal of potentially harmful medical or dental aerosols from a treatment space, such an operatory/hygiene room.

### BACKGROUND OF THE INVENTION

It has been known in the medical and dental industries that there is a need to protect personnel from harmful substances, such as noxious gases, infected body fluids, tissues and bone particulates that may be produced during medical and dental procedures. Certain known systems, such as U.S. Pat. No. 3,537,447, which is hereby incorporated by reference in its entirety, include a shielding apparatus that draws in air and filters the air in a unit mounted on the floor adjacent to the treatment seat. However, systems, such as the system of U.S. Pat. No. 3,537,447, do not provide adequate protection for the recent Severe Acute Respiratory Syndrome Coronavirus 2 (SARS-CoV-2), also known as COVID-19, which is known to be highly contagious from contact from infected bodily fluids. The exhaust of air, which has only been filtered, into the treatment space fails to provide sufficient protection for healthcare workers and for patients present in the space.

Dental procedures, such as drilling or cutting with dental tools, are common procedures for dentists and oral surgeons. Such procedures produce an aerosol of human tissue that becomes suspended in the air around the site of the procedures and which may contain disease, such as SARS-CoV-2, if the patient is infected. Such aerosols often include, for example, powdered bone or blood, both of which may carry SARS-CoV-2, if the patient is infected. Such aerosols can come into contact with mucus membranes of personnel in the operating field and may infect such personnel with the SARS-CoV-2 virus or other infectious diseases.

This is especially true with the use of dental scalers, scrapers, polishers, drills, and saws, particularly when used in conjunction with water jets, which produce a fine aerosol of fluids, tissue and bone chips, or may even cause a stream of blood to spurt into the air. Aerosolization of tissues or other particulates containing the SARS-CoV-2 may even occur during routine procedures, such as dental cleanings. Such aerosols remain in the air within the treatment space and may contaminate medical or dental practitioners in the room.

Potentially infectious aerosols, such as those formed during dental or other medical procedures, pose a danger to both medical personnel and patients. Currently, efforts to manage these aerosols are insufficient and do not reduce exposure sufficiently, are expensive and/or provide undesirable noise in operatory/hygiene rooms. A system and method for removal of potentially harmful aerosols from a treatment space that show one or more improvements in comparison to the prior art would be desirable in the art.

### SUMMARY OF THE INVENTION

The present invention relates to a system for removing potentially harmful aerosols for protecting medical or dental practitioners and patients from contaminants, including harmful aerosols created during medical and dental procedures emanated by patients into a treatment space. In particular, the system includes equipment positioned remote from the patient and the treatment space to remove the potentially harmful aerosols from the treatment space to a plenum space.

In one embodiment, a system for removing potentially harmful aerosols for protecting medical or dental practitioners and patients from contaminants, in accordance with the disclosure, includes a treatment space and a conduit extending from the treatment space to a plenum space. The conduit includes a suspended collection arm extending into the treatment space. The collection arm is movable to a position adjacent a source of aerosols. The system further includes an exhaust fan assembly arranged and disposed along the conduit to provide movement of air therethrough. An air treatment unit is arranged and disposed along the conduit to draw air through the conduit, filter the air and treat the air prior to discharging the air into the plenum space.

In another embodiment, a facility having a system for removing potentially harmful aerosols for protecting medical or dental practitioners and patients from contaminants, in accordance with the disclosure, includes a plurality of treatment spaces and an aerosol evacuation system. The aerosol evacuation system includes a conduit extending from each of the treatment spaces to a plenum space, each conduit having a suspended collection arm extending into the treatment space. The collection arm is movable to a position adjacent a source of aerosols. The system includes an exhaust fan assembly arranged and disposed along each conduit to provide movement of air therethrough. An air treatment unit is arranged and disposed to receive air from each conduit and to draw air through each conduit, filter the air and treat the air prior to discharging the air into the plenum space via a discharge.

In another embodiment a method for removing aerosols from a treatment space, in accordance with the disclosure, includes providing a conduit extending from a treatment space to a plenum space. The conduit includes a suspended collection arm extending into the treatment space. The method further includes moving the collection arm into a position adjacent a source of aerosols. Air is moved through the conduit away from the treatment space via an exhaust fan assembly arranged and disposed along the conduit. The air moving through the conduit is treated and filtered and discharged into the plenum space.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is a schematic view of an aerosol evacuation system, according to the embodiment of the present disclosure.

FIG. 2 is a schematic view of a collection arm and treatment space for the aerosol evacuation system shown in FIG. 1.

FIG. 3 is a schematic view of an air treatment unit for the aerosol evacuation system shown in FIG. 1.

FIG. 4 is a schematic view of an exhaust fan assembly for the aerosol evacuation system shown in FIG. 1.

Wherever possible, the same reference numbers will be used throughout the drawings to represent the same parts.

### DETAILED DESCRIPTION OF THE INVENTION

Provided is an aerosol evacuation system for removing potentially harmful aerosols for protecting medical or dental practitioners and patients from contaminants, including harmful aerosols created during medical and dental procedures emanated by patients into a treatment space. Embodiments of the present disclosure, for example, in comparison to concepts failing to include one or more of the features disclosed herein, reduce risk of dental staff and patients from being exposed to pathogens during patient care, provides little or no noise in the treatment space, as the air moving device is housed outside the treatment space, reduces maintenance by lowering the number of expensive filtration and treatment devices, and is modular such that the system provides ability to scale to a large number of treatments spaces. In addition, the system is unobtrusive in the treatment space, wherein the system does not take up floor space. The system, according to the present disclosure, permits evacuation of the potentially harmful aerosol utilizing a hands-free process.

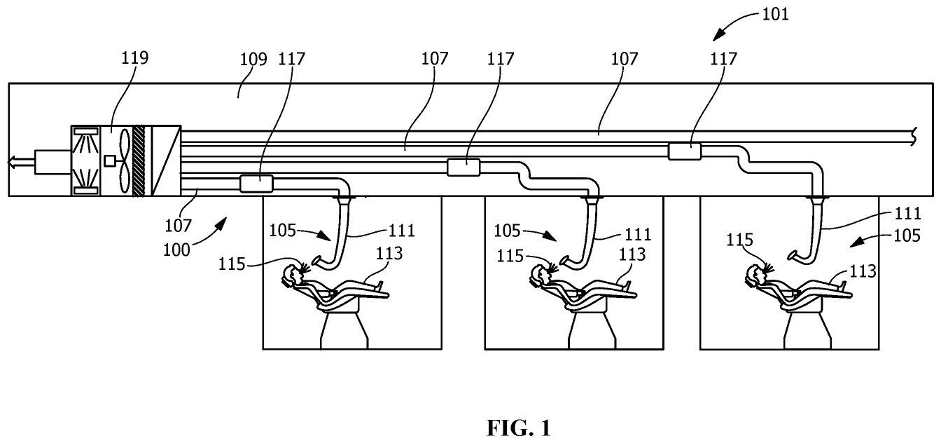

FIG. 1 shows an aerosol evacuation system 100 for reducing the potential for contamination of personnel during medical or dental procedures. The aerosol evacuation system 100 is part of a medical or dental facility 101 including a plurality of treatment spaces 105. The treatment spaces 105 are rooms or spaces in which patients are treated with medical or dental procedures. In one embodiment, the treatment space 105 is a dental treatment, hygiene or operatory room. The system 100 further includes a conduit 107 extending from the treatment space 105 to a plenum space 109. While the plenum space 109 in FIG. 1 is shown as a ceiling space, such as a ceiling space over a drop ceiling, the plenum space 109 may be any suitable space that is remote from the treatment space 105, such as a wall space, basement space or other space into which contaminated air may be discharged. For example, air may be discharged into any space where the air does not re-enter the treatment space 105 or other spaces occupied by patients or other personnel.

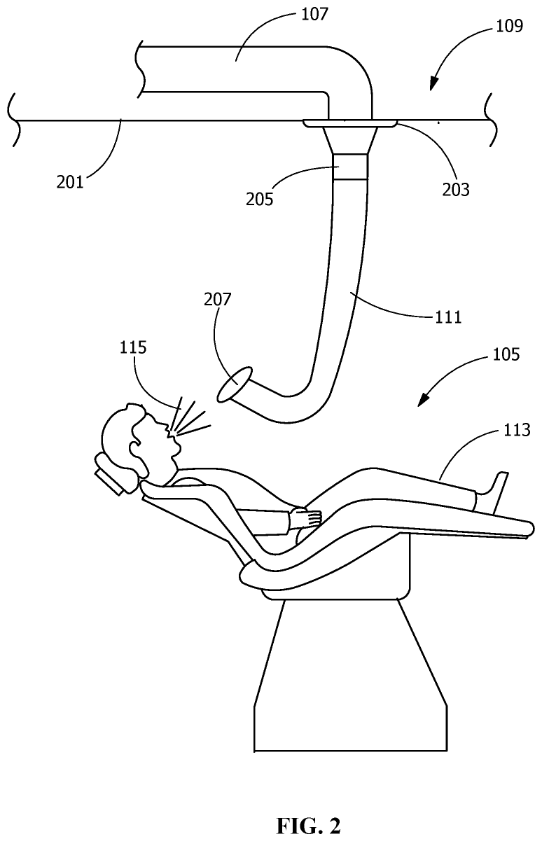

The conduit 107 includes a suspended collection arm 111 extending into the treatment space 105. The collection arm 111 is movable to a position adjacent a source of aerosols 115. As shown in FIGS. 1 and 2, the source of aerosols 115 is typically a patient 113. Patient 113 emanates aerosol 115 during medical or dental procedures, such as dental cleanings, drilling, scaping, scaling, sawing or other procedures that liberate patient tissues in a manner forming aerosolized tissues. In particular, aerosols 115 may be liberated during operation of dental equipment, such as scalers, saws or drills. The system according to the present disclosure is particularly suitable for aerosols 115 liberated from ultrasonic scalers and high-speed drills. As shown in FIG. 2, the collection arm 111 is suspended from ceiling 201 by ceiling flange 203. Ceiling flange 203 is any suitable structure that permits suspension of the collection arm 111 into treatment space 105 such that no floor space in treatment space 105 is required for the operation of system 100. In addition, collection arm 111 may optionally include a quick-release attachment 205, or other suitable fastening mechanism to permit removal of collection arm 111. In other embodiments, the collection arm 111 does not include a quick-release attachment 205, but may be otherwise mechanically engaged or engaged by fasteners such that the collection arm 111 may be removed manually or by utilizing tools. Removal of collection arm 111 permits replacement, servicing and/or cleaning of the collection arm 111. In one embodiment, the ceiling flange 203 is a track or slide to which the collection arm 111 is attached. The track or slide permits positioning of the collection arm 111 along the ceiling and within the treatment space 105 and allows passage of the aerosol 115 through the collection arm 111 into the conduit 107 in plenum space 109.

At the distal end of collection arm 111 an intake funnel 207 is provided to collect aerosol 115. Intake funnel 207 includes a flared end having increasing diameter extending from the collection arm 111 to receive the potentially harmful aerosols. The shape of the intake for the intake funnel 207 may include any suitable geometry for receiving aerosols 115. Suitable geometries for the cross-section of the intake funnel 207 may include square, rounded square, rectangle, rounded rectangle, oval, circular or other similar cross-sectional geometries that allow collection of aerosol 115 from patient 113. In one embodiment, the intake funnel 207 is an oval or rounded rectangular cross-section geometry having a first diameter of from 3 to 5 inches or about 4 inches, and a second diameter of from 7 to 9 inches or about 8 inches. The collection arm 111 may be any suitable structure that is capable of being positioned into and retained in a specific location. In one embodiment, the collection arm 111 is formed from a flexible, shape-holding material. For example, the collection arm 111 may include a segmented plastic arm having a plurality of engaged segments that permit individual movement with respect to the segments, allowing positioning and retention of the positioning. In another embodiment, collection arm 111 is a multi jointed arm permitting manual, flexible positioning of the intake funnel 207, while retaining the position after positioning. In still another embodiment, collection arm 111 is a fully flexible material that permits manual, selective positioning of the collection arm 111, while retaining the position after positioning. The positioning of collection arm 111 is such that medical or dental personnel may evacuate the potentially hazardous aerosols 115 hands-free or without manual manipulation once initially positioned. Suitable diameters for the collection arm 111 are any diameters capable of pulling aerosols from the aerosol source. For example, collection arm 111 may have an inner diameter of from 1 to 4 inches, or from 2 inches to 3 inches or about 2.75 inches.

In addition, system 100 includes an air treatment unit 119 and an exhaust fan assembly 117 arranged and disposed along the conduit to provide movement of air through conduit 107, filter the air and treat the air prior to discharging the air into the plenum space 109. As shown in FIG. 3, the air treatment unit 119 receives inlet air 301 at inlets 303. Although FIG. 3 shows four inlets 303, any number of inlets 303 may be utilized that permit filtering and treatment of the inlet air 301. In one embodiment, the air treatment unit 119 includes six, eight or ten inlets. Likewise, although FIG. 1 shows only a single air treatment unit 119, a plurality of air treatment units 119 may be utilized in order to filter and treat air from a greater number of treatment spaces 105. From inlets 303, the air passes over a negative pressure switch 305. The negative pressure switch 305 controls the exhaust fan assembly 117 in the event of loss of negative pressure to prevent reverse flow of air into the treatment space 105. In particular, the negative pressure switch 305 is a switch configured to monitor the pressure entering the air treatment unit and control and/or deactivate exhaust fan assemblies 117 in conduits 107 in the event that there is a loss of negative pressure. Alternatively, the negative pressure switch 305 may control and/or deactivate exhaust fan assemblies 117 in the event the pressure is sufficiently high to provide a risk of backflow of air into the treatment spaces 105. Although FIG. 3 shows the positioning of a negative pressure switch 305 at the inlet of air treatment unit 119, the invention is not so limited, and may include other arrangements of sensors and/or switches to monitor air pressure and prevent backflow of air into the treatment spaces 105. Primary fan 309 is independently activated and controlled and is activated when the system is in operation. In addition, primary fan 309 is sized such that the system, including all of the exhaust fan assemblies 117 may be activated simultaneously with the primary fan 309 maintaining a negative pressure at the negative pressure switch 305 of the air treatment unit 119.

From the negative pressure switch 305, the air passes through filter 307. In one embodiment, filter 307 is a high-efficiency particulate air (HEPA) filter. In another embodiment, filter 307 includes a pre-filter and a HEPA filter in series. The air is drawn through filter 307 via primary fan 309 then the air passes through a treatment chamber 311. Primary fan 309 may be any suitable air moving device, such as, but not limited to, a fan or blower. In one embodiment, the primary fan 309 is a variable speed fan blower. A single air moving device or multiple air moving devices may be utilized in air treatment unit 119. While not so limited, the primary fan 309 is capable of drawing air at a rate of 500 to 1500 cubic feet per minute or from 600 to 1200 cubic feet per minute within the air treatment unit 119. After the air is drawn though filter 307 by primary fan 309, the air is delivered to treatment chamber 311. UV lamp 313 is positioned within treatment chamber 311 to treat the air with UV radiation. Although the embodiment shown in FIG. 3 shows two UV lamps 313, a single lamp or any other suitable number of lamps may be used. In other embodiments, the treatment chamber 311 may include other treatment methods, such as ozone, radiation and/or chemical treatments. In one embodiment, either in conjunction with the UV radiation or in place of the UV radiation, the air treatment unit 119 includes an ozone generator to release ozone and treat the air passing through the air treatment unit. After treatment, discharge air 315 is discharged via discharge 317 into plenum space 109 (as shown in FIG. 1).

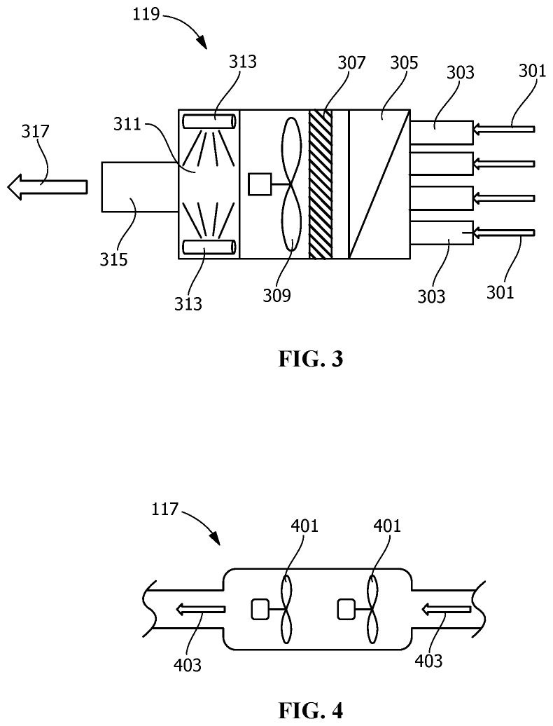

FIG. 4 shows exhaust fan assembly 117, according to an embodiment of the present disclosure. The exhaust fan assembly 117 includes fans 401 that are activatable to move air 403 from treatment space 105 to the air treatment unit 119. In one embodiment, fans 401 may be activated by personnel in treatment space 105 to selectively provide evacuation of the aerosol. For example, the fans 401 may be activated to remove aerosol only during certain procedures, while fans 401 may be deactivated when aerosol evacuation is not required. While FIG. 4 shows two fans 401 in the exhaust fan assembly 117, the invention is not so limited and may include a single fan 401 or any other suitable number of fans in exhaust fan assembly 117 that provide the desired airflow from the treatment space 105.

As shown in FIG. 1, embodiments of the present disclosure may include a medical or dental facility made up of a plurality of treatment spaces having the aerosol evacuation system 100, according to the present disclosure. The aerosol evacuation system 100 is modular, permitting flexibility to provide packages that serve a single room or a plurality or rooms. For example, in one embodiment, the system 100 may allow for the one to four rooms per air treatment unit 119 or five to eight rooms per air treatment unit 119.

In one embodiment, the invention includes a method for removing aerosols from a treatment space 105. The method utilizes conduit 107 extending from a treatment space 105 to plenum space 109 (see, for example, FIG. 1), the conduit 107 includes suspended collection arm 111 extending into the treatment space 105. In this method the medical or dental practitioner may manually move the collection arm 111 into a position adjacent a potential source of aerosols. Once in position, the collection arm 111 remains in position during the procedure. Generally, the positioning corresponds to the site of a medical or dental procedure. For example, in the instance where the procedure is a dental procedure, the collection arm 111 is brought into a position near the mouth of the patient or the source or the aerosol 115. In one embodiment, the collection arm 111 and the intake funnel 207, such that the intake portion of the intake funnel 207 is positioned at a distance of 5 inches to 10 inches or 6 inches to 9 inches or 7 inches to 8 inches from the source of aerosol 115. After the collection arm 111 is positioned, exhaust fan assembly 117 is activated to move air through conduit 107 away from treatment space 105. Air is delivered to air treatment unit 119, where the air is filtered, treated and discharged into plenum space 109.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An aerosol evacuation system for reducing the potential for contamination of personnel during medical or dental procedures, the system comprising:
a treatment space;
a conduit extending from the treatment space to a plenum space, the conduit including a suspended collection arm extending into the treatment space, the collection arm being movable to a position adjacent a source of aerosols;
an exhaust fan assembly is arranged and disposed along the conduit to provide movement of air therethrough; and
an air treatment unit arranged and disposed along the conduit to draw air through the conduit, filter the air and treat the air prior to discharging the air into the plenum space.

2. The system of claim 1, wherein the collection arm is formed from a flexible, shape-holding material.

3. The system of claim 1, wherein the collection arm is suspended from a ceiling flange.

4. The system of claim 3, wherein the collection arm includes a quick-release attachment to release from the ceiling flange for cleaning or replacement.

5. The system of claim 1, wherein the air treatment unit includes a UV light to treat the air prior to discharging the air.

6. The system of claim 1, wherein the air treatment unit includes a HEPA filter to filter the air prior to discharging the air. The system of claim 1, wherein the air treatment unit includes a capacity of from 600 to 1200 cubic feet per minute of airflow.

8. The system of claim 1, wherein the air treatment unit includes a negative pressure switch configured to control the exhaust fan to prevent airflow into the treatment space through the conduit.

9. The system of claim 1, wherein the air treatment unit receives air from a plurality of conduits.

10. The system of claim 1, wherein the system is retrofit into an existing treatment space and plenum space.

11. A medical or dental facility comprising:
a plurality of treatment spaces;
an aerosol evacuation system comprising:
a conduit extending from each of the treatment spaces to a plenum space, each conduit including a suspended collection arm extending into the treatment space, the collection arm being movable to a position adjacent a source of aerosols;
an exhaust fan assembly is arranged and disposed along each conduit to provide movement of air therethrough; and
an air treatment unit arranged and disposed to receive air from each conduit and to draw air through each conduit, filter the air and treat the air prior to discharging the air into the plenum space via a discharge.

12. The facility of claim 11, wherein each collection arm is formed from a flexible, shape-holding material.

13. The facility of claim 11, wherein each collection arm is suspended from a ceiling flange.

14. The facility of claim 13, wherein each collection arm includes a quick-release attachment to release from the ceiling flange for cleaning or replacement.

15. The facility of claim 11, wherein the air treatment unit includes a UV light to treat the air prior to discharging the air.

16. The facility of claim 11, wherein the air treatment unit includes a HEPA filter to filter the air prior to discharging the air.

17. The facility of claim 11, wherein the air treatment unit includes a capacity of from 600 to 1200 cubic feet per minute of airflow.

18. The facility of claim 11, wherein the air treatment unit includes a negative pressure switch configured to control the exhaust fan to prevent airflow into the treatment space through the conduit.

19. A method for removing aerosols from a treatment space, the method comprising:
providing a conduit extending from a treatment space to a plenum space, the conduit including a suspended collection arm extending into the treatment space;
moving the collection arm into a position adjacent a source of aerosols;
moving air through the conduit away from the treatment space via an exhaust fan assembly arranged and disposed along the conduit; and
treating and filtering the air moving through the conduit;
discharging the air in the plenum space.
